(19) United States
(12) Patent Application Publication
(10) Pub. No.: US 2023/0008788 A1
(43) Pub. Date: Jan. 12, 2023

(54) Point of Care Claim Processing System and Method

(71) Applicant: Overjet, Inc., Boston, MA (US)

(72) Inventors: Wardah Inam, Boston, MA (US); Deepak Ramaswamy, Newton, MA (US); Patrick Austermann, Princeton, NJ (US); James Russo, Bella Collina, FL (US); Gilad Sherry-Musafi, Boston, MA (US); Siddharth V. Balwani, San Jose, CA (US); Thomas Patrick Keane, East Walpole, MA (US)

(21) Appl. No.: 17/863,269

(22) Filed: Jul. 12, 2022

Related U.S. Application Data
(60) Provisional application No. 63/220,812, filed on Jul. 12, 2021.

(51) Int. Cl. G06Q 40/08; G16H 50/20; G16H 30/40

(52) U.S. Cl. CPC G06Q 40/08; G16H 50/20; G16H 30/40

(57) ABSTRACT

A computer-implemented method and system provide point of care processing of an insurance claim relating to oral care delivered to a subject patient during a visit of the patient to a dental clinic. The method includes processing, by a computer system, of dental image data and patient data, using a set of machine learning models, to extract output representative of diagnostic data characterizing the dental image data; determining, by a decision support system a claim decision based on the diagnostic data; and communicating the claim decision in real time to an endpoint located in the dental clinic.

### PRIORITY

The present patent application claims priority from U.S. provisional patent application Ser. No. 63/220,812, filed Jul. 12, 2021. This Application is hereby incorporated herein, in its entirety, by reference.

### TECHNICAL FIELD

The present invention relates to point of care claim processing, and more particularly to processing of claims relating to provision of dental services.

### BACKGROUND ART

Dental insurance claims and requests for treatment approval (or pre-approval) require careful analysis of the supporting materials submitted by dental-care providers (or other types of health providers). Given the large volume of such claims, traditional claim processing requires many reviewers to assess submitted claims. This review process often results in inconsistent decision-making by different reviewers, and in errors in insurance decisions. Errors are also caused by the challenge in accurately assessing a large volume of materials accompanying each claim. The review process, even when expedited, can rarely be completed in less than a few hours, much less within a few minutes after submission of a claim or a request for pre-approval from a provider.

### SUMMARY OF THE EMBODIMENTS

In accordance with one embodiment of the invention, there is provided a computer-implemented method for point of care processing of an insurance claim relating to oral care delivered to a subject patient during a visit of the patient to a dental clinic. The method of this embodiment utilizes a computer system executing instructions establishing computer processes, and the computer processes include:

receiving, by the computer system, (i) dental image data, pertaining to the subject patient, obtained from a diagnostic imaging system located in a dental clinic and (ii) patient data maintained for the subject patient;

processing, by the computer system, the dental image data and at least some of the patient data, using a set of machine learning models, to extract output representative of diagnostic data characterizing the dental image data;

determining, by a decision support system, applicable to an entity selected from the group consisting of a pertinent insurance payer, a provider, a patient plan, and combinations thereof, a claim decision based on the diagnostic data; and

communicating the claim decision in real time to an endpoint located in the dental clinic.

Optionally, the determining by the decision support system includes making a determination whether to provide pre-authorization for an oral care procedure. Also optionally, the determining by the decision support system includes determining, by a rule engine applying rules of a rule set selected for applicability to the entity, the claim decision based on the diagnostic data.

Optionally, the determining by the decision support system includes determining, by a machine learning system, the claim decision based on the diagnostic data.

Also optionally, the receiving the patient data includes receiving information selected from the group consisting of (a) patient demographics, (b) subscriber demographics for the patient, (c) a proposed treatment plan for the patient, (d) periochart data, (e) previously completed treatments, (f) patient health history, (g) patient medication list, and combinations thereof. Optionally, the patient demographics are selected from the group consisting of patient name, patient date of birth, patient id number, patient relationship to a subscriber, and combinations thereof

In accordance with another embodiment of the present invention, there is provided a computer-readable non-transitory storage medium storing instructions that, when executed by a computer system, establish computer processes, for point of care processing of an insurance claim relating to oral care delivered to a subject patient during a visit of the patient to a dental clinic, wherein the processes comprise the processes recited above in connection each of the foregoing methods.

### BRIEF DESCRIPTION OF THE DRAWINGS

The foregoing features of embodiments will be more readily understood by reference to the following detailed description, taken with reference to the accompanying drawings, in which:

FIG. 1 is a block diagram of a point of care adjudication system, in accordance with an embodiment of the present invention, shown with associated sources of input data and rules data, and also showing the output arrangement.

FIG. 2 is an example of a result code table showing codes for an output of the point of care adjudication system of FIG. 1, as made available at a point of care of a patient, in accordance with an embodiment of the present invention.

FIG. 3 is an example of a set of rules that can be employed in a rule-based decision support system in accordance with an embodiment of the present invention.

FIG. 4 is a block diagram of a machine learning system for use in accordance with an embodiment of the present invention.

FIG. 5 is a generalized block diagram of a point of care adjudication system in accordance with an embodiment of the present invention.

### DETAILED DESCRIPTION OF SPECIFIC EMBODIMENTS

Definitions. As used in this description and the accompanying claims, the following terms shall have the meanings indicated, unless the context otherwise requires:

A “computer process” is the performance of a described function in a computer system using computer hardware (such as a processor, field-programmable gate array or other electronic combinatorial logic, or similar device), which may be operating under control of software or firmware or a combination of any of these or operating outside control of any of the foregoing. All or part of the described function may be performed by active or passive electronic components, such as transistors or resistors. In using the term “computer process,” we do not necessarily require a schedulable entity, or operation of a computer program or a part thereof, although, in some embodiments, a computer process may be implemented by such a schedulable entity, or operation of a computer program or a part thereof. Furthermore, unless the context otherwise requires, a “process” may be implemented using more than one processor or more than one (single- or multi-processor) computer.

A “set” includes at least one member.

“Point of care processing” refers to performing a process at a point of care such as a dental clinic.

A “diagnostic imaging system” is a device that provides a digital image output relating to an oral cavity of a patient

An “oral cavity” of a patient is the patient's mouth. It includes the lips, the lining inside the cheeks and lips, the front two thirds of the tongue, the upper and lower gums, the floor of the mouth under the tongue, the bony roof of the mouth, and the small area behind the wisdom teeth.

A “dental clinic” or “point of care” is a physical location in which oral care services are performed.

“Patient data” includes data about a subject patient. It includes demographic information such as address or date of birth, past, present or future claims or medical or dental conditions, diagnostic information such as narratives or radiographs, consent, treatment plan or notes.

A “claim decision” includes a determination selected from the group consisting of an adjudication, a pre-authorization, and an approval.

A claim decision concerning a patient is communicated “in real time” to an endpoint in a dental clinic if it is communicated in the course of a visit by the patient to the dental clinic.

“Subscriber demographics” for a patient includes information identifying a subscriber to an insurance plan potentially applicable to the patient and related information about the subscriber and the plan.

An “endpoint” in a dental clinic is a node having a display located in the dental clinic.

A “decision support system” is an information system that supports decision-making activities. Examples of such an information system include a machine learning system and a rule evaluation system.

“Pre-authorization” of an oral care procedure for a subject patient is a decision that a payer will likely accept a claim for reimbursement for performing the oral care procedure.

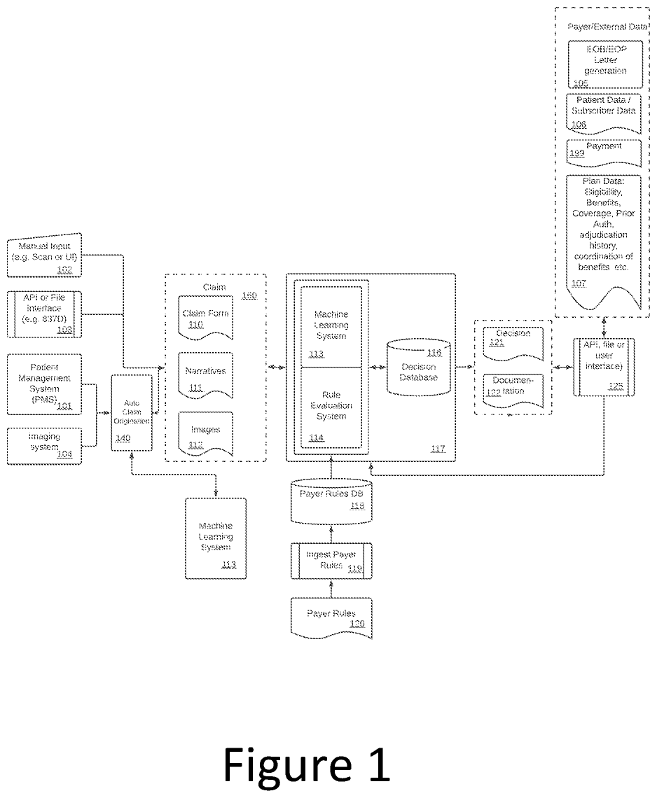

FIG. 1 is a block diagram of a point of care adjudication system in accordance with an embodiment of the present invention, shown with associated sources of input data and rules data, and also showing the output arrangement. In this embodiment, a practice management system (PMS) 101 and imaging system 104 provide textual and image data used by the auto-claim origination system 140 to generate automatically a claim 160 based on a proposed plan of treatment. The auto-claim origination system 140 is support by machine learning system 113 that evaluates image data from the imaging system 104 and textual data from patient management system 101. Claim 160 is defined by a claim form 110, narratives 111, and images 112. Components of the claim 160 may optionally be defined, at least in part, from Manual input 102, such as a scan or user interface (UI), or by API or file interface 103.

Also in FIG. 1, the point of care adjudication system 117 receives data characterizing the claim 160 as an input, and after processing of the claim may cause some or all of its components to be updated. The point of care adjudication system 117 uses machine learning system 113 (which may be the same or another instance of machine learning system 113 used by the auto-claim origination system 140) to process image data and other related data to evaluate the claim 160. The point of care adjudication system 117 also applies payer rules 120 from payer rules database 118 via rule evaluation system 114. The Payer rules database 118 in turn is developed by ingesting rules processor 119, which has payer rules 120 as an input thereto. The point of care adjudication system 117 reads data from, and writes data to, decision database 116 to produce decision 121 and associated documentation 122. The decision 121 and documentation 122 are made available to the payer through an appropriate bidirectional API, file, or user interface 125. In turn, the payer makes available via the API, file, or user interface 125 items including Explanation of Benefits (EOB) / Explanation of Payment EOP letter generation 105, patient/subscriber data 106, payment 199, and information 107 including plan data, eligibility, benefits, coverage, prior authentication, adjudication history, coordination of benefits, etc. Because information flow over item 125 is bidirectional, it is also available for use in further processing by the point of care adjudication system 117.

Machine learning system 113 of FIG. 1 may be implemented as a neural network. Such neural networks may be realized using different types of neural network architectures, configuration, and/or implementation approaches. Examples of neural networks that may be used include a convolutional neural network (CNN), a feed-forward neural network, a recurrent neural network (RNN), a transformer network, etc. Feed-forward networks include one or more layers of nodes (“neurons” or “learning elements”) with connections to one or more portions of the input data. In a feedforward network, the connectivity of the inputs and layers of nodes is such that input data and intermediate data propagate in a forward direction towards the network's output. There are typically no feedback loops or cycles in the configuration / structure of the feed-forward network. Convolutional layers allow a network to efficiently learn features by applying the same learned transformation(s) to subsections of the data. A transformer network is a machine learning configuration (used, for example, in natural language processing and computer vision applications) that includes an attention mechanism to weight network connections according to their significance. Other examples of learning engine approaches / architectures that may be used include generating an auto-encoder and using a dense layer of the network to correlate with probability for a future event through a support vector machine, constructing a regression or classification neural network model that indicates a specific output from data (based on training reflective of correlation between similar records and the output that is to be identified), etc.

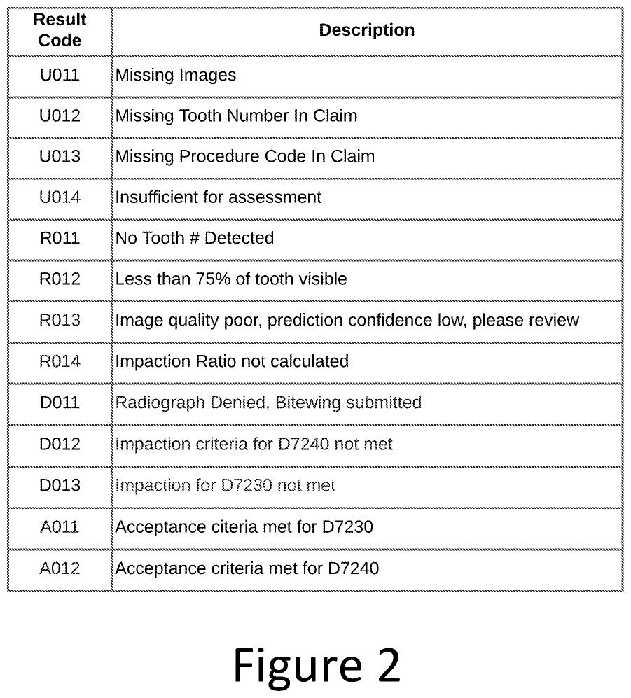

FIG. 2 is a result code table showing an example of codes for a decision output of the point of care adjudication system of FIG. 1, as made available at a point of care of a patient, in accordance with an embodiment of the present invention. These result codes describe, for example, whether a claim was accepted, denied, or not decided due to missing information. The decision support system, such as rule evaluation system 114, will determine these result codes. If the acceptance criteria in the processing tree have been met, result codes such as A011 and A012 are returned. Decision codes such as U011, U012, U013 or U014 express the lack of sufficient information to render a decision on this claim. Codes such as D011, D012, or D13 signify a denial of a claim. Codes such as R011, R012, R013 and R014 express that a decision could not be made by the process and requires further review.

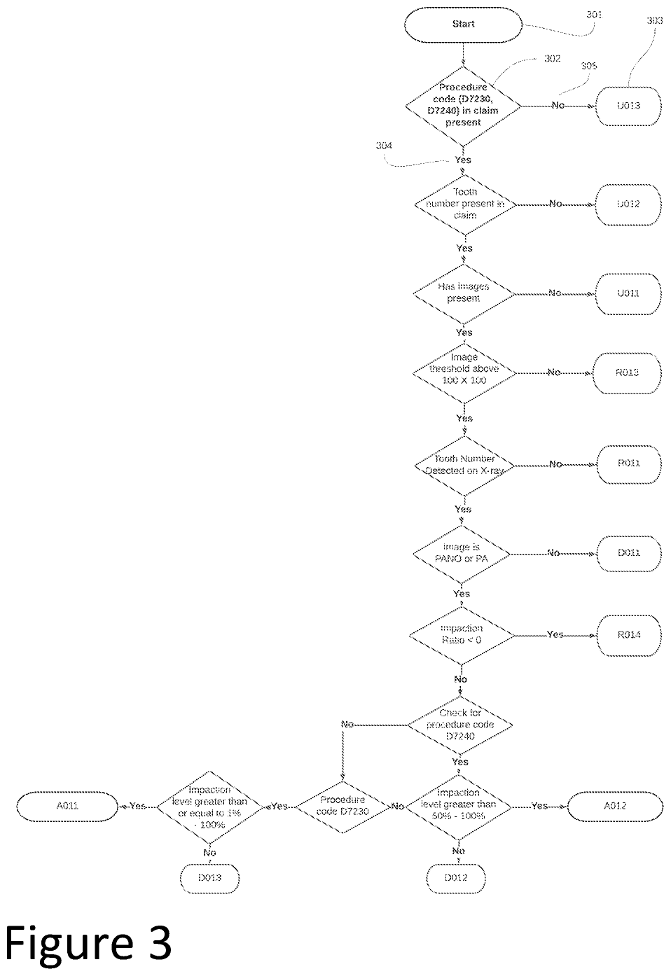

FIG. 3 is an example of a set of rules that can be employed in a rule-based decision support system, including the rule evaluation system 114 of FIG. 1, in accordance with an embodiment of the present invention. Evaluation of a claim begins at the start node 301. The evaluation follows a sequence of decisions such as 302, where each successive step requires evaluation of a further condition. When the outcome of an evaluation is in the affirmative, the logical flow follows the path of the “Yes” arrow (such as arrow 304) or, if the outcome of the evaluation is negative, logical flow follows the path of the “No” arrow (such as arrow 305). A negative evaluation terminates the logical flow, such as at decision node 303, for which is produced a result code, in this case U013. In FIG. 2, we reproduce a table of typical result codes.

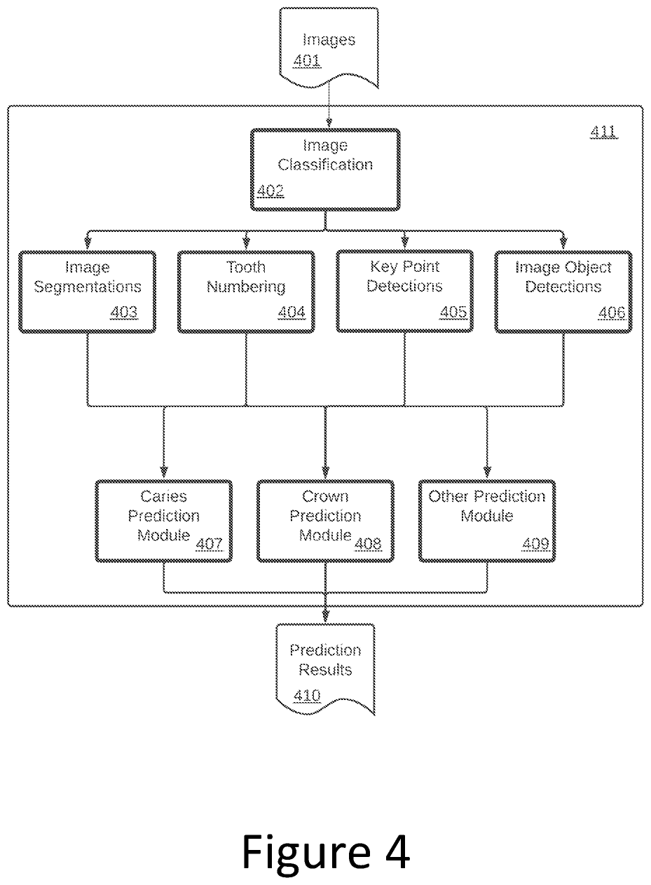

FIG. 4 is a block diagram of a machine learning system 411 that may be used, in accordance with an embodiment of the present invention, for example, as the machine learning system 113 of FIG. 1 to analyze images 401, which may be radiographs. Items making up machine learning system 411 include a fixed or variable sequence of processing stages. In one embodiment, image classification stage 402 classifies an image into different classes to determine whether they are radiographs and, if so, what type of radiographs they are. Radiograph types include bitewing, periapical, occlusal, and panoramic radiographs and three-dimensional images originating from cone-beam computed tomography systems. The image class determines the subsequent analysis stages. Image segmentations 403 determine regions of interest on a radiograph such as the outline of a tooth. Tooth numbering 404 associates a standardized identifier for each tooth visible on the radiograph, such as “9” for the upper left central incisor tooth. Key point detection 405 identifies important anatomical locations on the radiograph, such as the tip of the root of a tooth. Additional features of the radiograph may be detected in image object detections 406. Following one or more of the foregoing stages, clinical conditions are detected by caries prediction module 407, crown prediction module 408, and other prediction module 409. The machine learning system 411 combines results from previous stages and provides as an output the aggregated prediction results 410 for further processing.

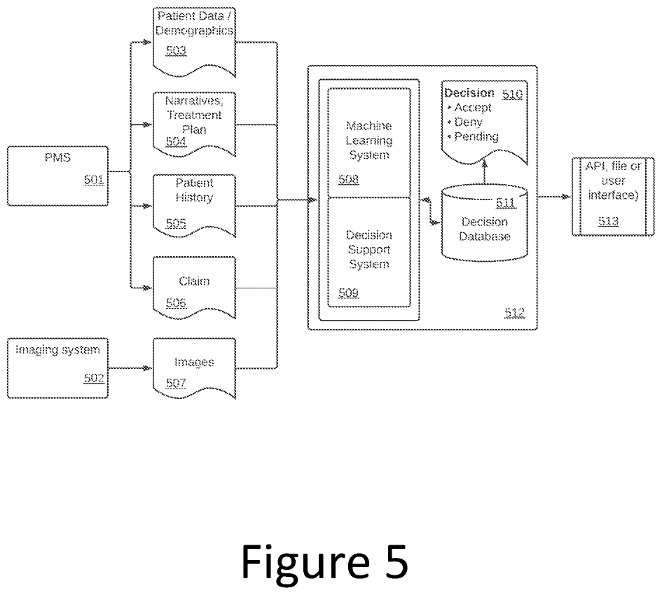

FIG. 5 is a block diagram of a point of care adjudication system 512, in accordance with an embodiment of the present invention, which has been generalized from the diagram of FIG. 1. The system includes practice management system 501 having components including textual data relating to patient data/demographics 503, narratives and treatment plan 504, patient history 505, and claim 506 for which a claim decision is solicited. Also pertinent to the point of care are images 507 originating from imaging system 502. The point of care adjudication system 512 has elements including a machine learning system 508 to process the data including image data 507 and textual data items 503, 504, 505, and 506. The machine learning system 508 is described in more detail in FIG. 4. The adjudication system 512 reads data from and writes data to a decision database 511, wherein the decision 510 may be to accept, deny, or to maintain a claim as pending. The decision is then provided to another computer process 513, which may include a user interface (UI), application programming interface (API), file or other output system.

Implementations described herein, including implementations using neural networks, can be realized on any computing platform, including computing platforms that include one or more microprocessors, microcontrollers, and/or digital signal processors that provide processing functionality, as well as other computation and control functionality. The computing platform can include one or more CPU's, one or more graphics processing units (GPU's, such as NVIDIA GPU's), and may also include special purpose logic circuitry, e.g., an FPGA (field programmable gate array), an ASIC (application-specific integrated circuit), a DSP processor, an accelerated processing unit (APU), an application processor, customized dedicated circuit, etc., to implement, at least in part, the processes and functionality for the neural networks, processes, and methods described herein. The computing platforms typically also include memory for storing data and software instructions for executing programmed functionality within the device. Generally speaking, a computer accessible storage medium may include any non-transitory storage media accessible by a computer during use to provide instructions and/or data to the computer. For example, a computer accessible storage medium may include storage media such as magnetic or optical disks and semiconductor (solid-state) memories, DRAM, SRAM, etc. The various learning processes implemented through use of the neural networks may be configured or programmed using PyTorch or TensorFlow (a software library used for machine learning applications such as neural networks). Other programming platforms that can be employed include keras (an open-source neural network library) building blocks, NumPy (an open-source programming library useful for realizing modules to process arrays) building blocks, etc.

Although particular embodiments have been disclosed herein in detail, this has been done by way of example for purposes of illustration only, and is not intended to be limiting with respect to the scope of the appended claims, which follow. Any of the features of the disclosed embodiments can be combined with each other, rearranged, etc., and are within the scope of the invention to produce more embodiments. Some other aspects, advantages, and modifications are considered to be within the scope of the claims provided below. The claims presented are representative of at least some of the embodiments and features disclosed herein. Other unclaimed embodiments and features are also contemplated.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A computer-implemented method for point of care processing of an insurance claim relating to oral care delivered to a subject patient during a visit of the patient to a dental clinic, the method utilizing a computer system executing instructions establishing computer processes, the computer processes comprising:
receiving, by the computer system, (i) dental image data, pertaining to the subject patient, obtained from a diagnostic imaging system located in a dental clinic and (ii) patient data maintained for the subject patient;
processing, by the computer system, the dental image data and at least some of the patient data, using a set of machine learning models, to extract output representative of diagnostic data characterizing the dental image data;
determining, by a decision support system, applicable to an entity selected from the group consisting of a pertinent insurance payer, a provider, a patient plan, and combinations thereof, a claim decision based on the diagnostic data; and
communicating the claim decision in real time to an endpoint located in the dental clinic.

2. A computer-implemented method according to claim 1, wherein the determining by the decision support system includes making a determination whether to provide pre-authorization for an oral care procedure.

3. A computer-implemented method according to claim 1, wherein the determining by the decision support system includes determining, by a rule engine applying rules of a rule set selected for applicability to the entity, the claim decision based on the diagnostic data.

4. A computer-implemented method according to claim 1, wherein the determining by the decision support system includes determining, by a machine learning system, the claim decision based on the diagnostic data.

5. A computer-implemented method according to claim 1, wherein the receiving the patient data includes receiving information selected from the group consisting of (a) patient demographics, (b) subscriber demographics for the patient, (c) a proposed treatment plan for the patient, (d) periochart data, (e) previously completed treatments, (f) patient health history, (g) patient medication list, and combinations thereof.

6. A computer-implemented method according to claim 5, wherein the patient demographics are selected from the group consisting of patient name, patient date of birth, patient id number, patient relationship to a subscriber, and combinations thereof.

7. A computer-readable non-transitory storage medium storing instructions that, when executed by a computer system, establish computer processes, for point of care processing of an insurance claim relating to oral care delivered to a subject patient during a visit of the patient to a dental clinic, wherein the processes comprise:
receiving, by the computer system, (i) dental image data, pertaining to the subject patient, obtained from a diagnostic imaging system located in a dental clinic and (ii) patient data maintained for the subject patient;
processing, by the computer system, the dental image data and at least some of the patient data, using a set of machine learning models, to extract output representative of diagnostic data characterizing the dental image data;
determining, by a decision support system , applicable to an entity selected from the group consisting of a pertinent insurance payer, a provider, a patient plan, and combinations thereof, a claim decision based on the diagnostic data; and
communicating the claim decision in real time to an endpoint located in the dental clinic.

8. A computer-readable non-transitory storage medium according to claim 7, wherein the determining by the decision support system includes making a determination whether to provide pre-authorization for an oral care procedure.

9. A computer-readable non-transitory storage medium according to claim 7, wherein the determining by the decision support system includes determining, by a rule engine applying rules of a rule set selected for applicability to the entity, the claim decision based on the diagnostic data.

10. A computer-readable non-transitory storage medium according to claim 7, wherein the determining by the decision support system includes determining, by a machine learning system, the claim decision based on the diagnostic data.

11. A computer-readable non-transitory storage medium according to claim 7, wherein the receiving the patient data includes receiving information selected from the group consisting of (a) patient demographics, (b) subscriber demographics for the patient, (c) a proposed treatment plan for the patient, (d) periochart data, (e) previously completed treatments, (f) patient health history, (g) patient medication list, and combinations thereof.

12. A computer-readable non-transitory storage medium according to claim 11, wherein the patient demographics are selected from the group consisting of patient name, patient date of birth, patient id number, patient relationship to a subscriber, and combinations thereof

13. A computer-readable non-transitory storage medium according to claim 1, wherein the computer processes further comprise receiving, from the pertinent insurance payer, data relating to the claim decision and updating the claim decision in response thereto.

14. A computer-readable non-transitory storage medium according to claim 1, wherein the computer processes further comprise automatically generating a claim as a result of processing of the dental image data and the patient data, such claim being made subject to processing by the decision support system.
